(19) United States
(12) Patent Application Publication
(10) Pub. No.: US 2016/0008217 A1
(43) Pub. Date: Jan. 14, 2016

(54) VIBRATIONAL WOUND DRESSING AND METHOD OF USE

(71) Applicant: WOUND MANAGEMENT TECHNOLOGIES, INC., Fort Worth, TX (US)

(72) Inventor: Barry Everett Constantine, Island Heights, NJ (US)

(21) Appl. No.: 14/328,356

(22) Filed: Jul. 10, 2014

(51) Int. Cl. A61H 23/00; A61F 13/00

(52) U.S. Cl. CPC A61H 23/00; A61F 13/00004

(57) ABSTRACT

A wound dressing, comprising one or more vibrational devices configured to generate a mechanical vibration to stimulate a wound. A controller coupled to the one or more vibrational devices and configured to activate, deactivate and/or control as may be programmed in the vibrational devices. A primary and secondary bandage coupled to the vibrational devices and configured to secure the vibrational devices adjacent to the wound associated area.

### TECHNICAL FIELD

The present disclosure relates generally to wound care, and more specifically to a vibrational wound dressing and method of use that accelerates healing.

### BACKGROUND OF THE INVENTION

Wound dressings are designed to immobilize a wound, to prevent it from further movement and potential damage.

### SUMMARY OF THE INVENTION

A wound dressing, comprising one or more vibrational devices configured to generate a mechanical vibration to stimulate a wound. A controller coupled to the one or more vibrational devices and configured to activate and deactivate the vibrational devices ad libitum and can be programmable. A bandage coupled to the vibrational devices and configured to secure the vibrational devices adjacent to the wound as a primary dressing or as a removable, non-wound contact, secondary dressing. The vibrational device powered by battery, solar energy source, generator, alternator or household current.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views, and in which:

FIG. 1 is a diagram of a system for providing vibrational dressing in accordance with an exemplary embodiment of the present disclosure;

FIG. 2 is a diagram of a controller for a vibrational wound dressing, in accordance with an exemplary embodiment of the present disclosure; and

FIG. 3 is a diagram of an algorithm in accordance with an exemplary embodiment of the present disclosure.

### DETAILED DESCRIPTION OF THE INVENTION

In the description that follows, like parts are marked throughout the specification and drawings with the same reference numerals. The drawing figures might not be to scale and certain components can be shown in generalized or schematic form and identified by commercial designations in the interest of clarity and conciseness.

It has been observed that wounds exposed to vibration formed more granulation tissue, a type of tissue important early in the wound-healing process. The vibration also appeared to help tissue to form new blood vessels through a process called angiogenesis, and also appeared to lead to increased expression of pro-healing growth factors and signaling molecules called chemokines. It has also been observed that wounds exposed to vibration five times a week for 30 minutes healed more quickly than wounds in mice of a control group. However, continual or near-continual vibration has never been attempted.

The combination of a controlled source of vibrational energy with a primary wound dressing will keep the biological elements within the wound space in a form of enhanced Brownian motion facilitating biochemical interaction with the wound and surrounding tissue. The controlled source of vibrational energy can be provided through a secondary, non-wound contact, wound dressing which overlays a primary wound dressing designed to remain in place over an extended period, and can provide vibrational energy to the wound ad libitum.

One measurement of vibrational acceleration in meters per second squared (m/s²). The vibrational exposure direction is also important to control, and can be measured in a well-defined direction.

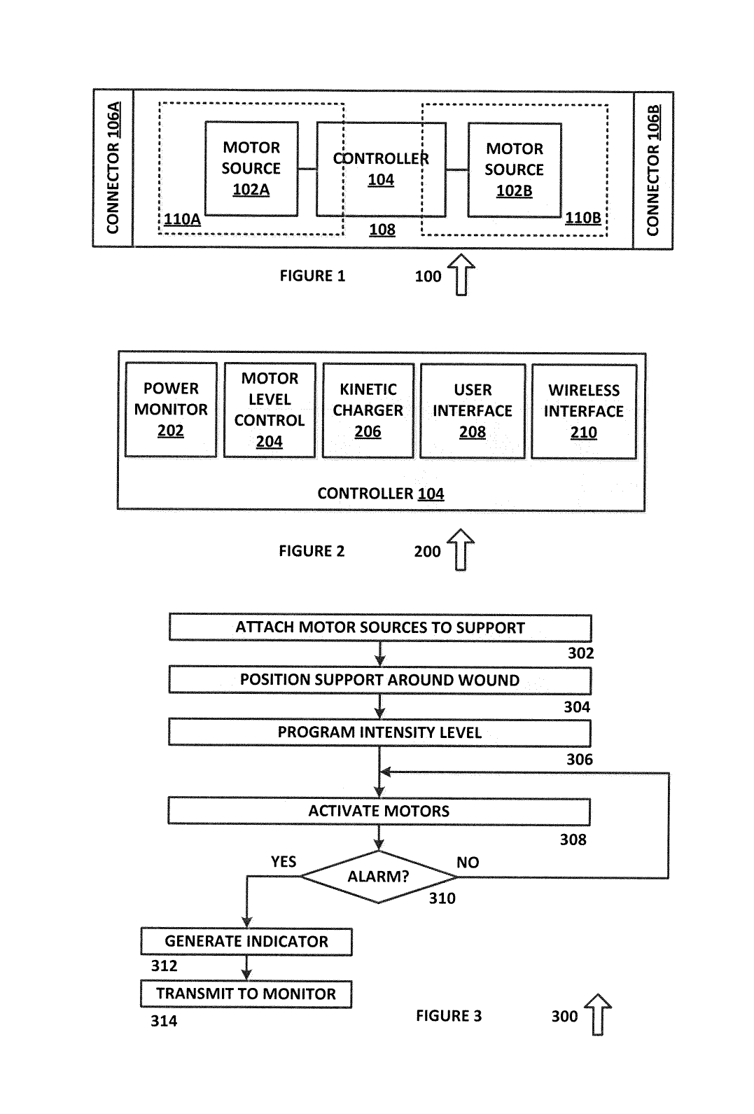

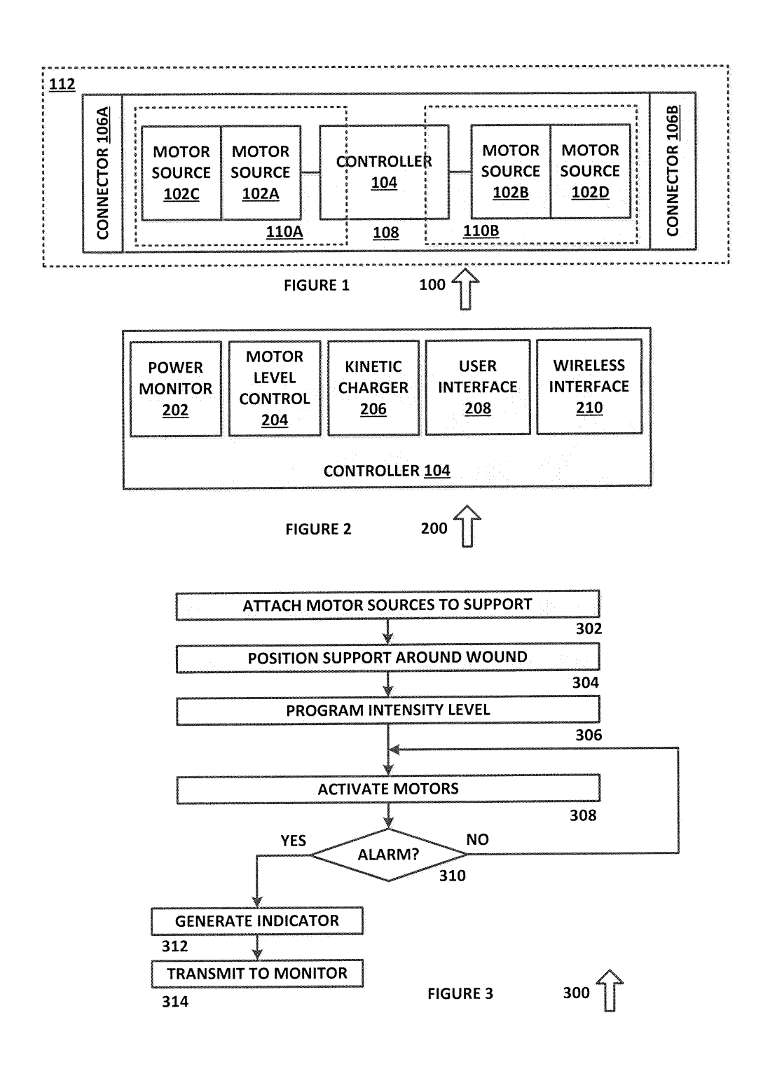

FIG. 1 is a diagram of a system 100 for providing vibrational dressing in accordance with an exemplary embodiment of the present disclosure. System 100 can be implemented in hardware or a suitable combination of hardware and software, and can be one or more software systems operating on a processor and associated devices.

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes a microcomputer or other suitable controller, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections.

Chronic wounds result when local circulation is cut off, either by external pressure or intravascular fibrin clots. Chronic wounds which result from external pressure are addressed by rotation of the body or physical obstruction (such as from a stone in a shoe for example). The body's mechanism to resolve intravascular fibrin clots involves the release of Plasminogen Activator from the adjacent endovascular endothelium, the blood vessel cells that line blood vessels, adjacent to the clot. Plasminogen Activator reacts with plasminogen in the circulation to form plasmin, a fibrin clot digesting enzyme whose function is to digest the intravascular fibrin clot and restore circulation to the site such that healing can proceed.

Active wound dressing techniques being employed today include the introduction of autologous growth factors, exogenous growth factors which have not proven to exceed the rate of healing compared to occluded controls.

Negative pressure has been shown to promote healing of chronic wounds as the atmospheric pressure surrounding the associated blood vessels is reduced to the point that vascular pores are enlarged allowing in the flow of elements needed for wound healing to proceed. The success of negative pressure demonstrates the efficacy of stimulating an indolent vasculature to one that contributes to healing. Negative pressure does not affect intravascular clot formation which is responsible for the re occurrence of chronic wounds. Once the negative pressure is removed vascular pores close and unless the intravascular fibrin clot is dislodged, the wound ultimately reoccurs.

The vibration dressing mechanism of the present disclosure provides a constant physical vibration to the periwound area wherein the vasculature is obstructed by these intravascular fibrin clots. Extracorporeal vibration is expected to encourage the release of the autologous Plasminogen Activator (PA) from the local endothelial vasculature, via tissue vibration and clot to endothelial interaction, causing chemical reaction of PA with plasminogen to form autologous plasmin which digests the adjacent intravascular fibrin clot.

System 100 includes stimulation motor sources 102A and 102B, which can be cell phone vibrator motors, pager vibrator motors or other suitable vibrating devices. Controller 104 is coupled to motor sources 102A and 102B, which have an associated affected area 110A and 110B, respectively, that has a size that can be a function of an amount of vibrational energy that is generated by each motor source, and can be disposed on bandage 108 or can otherwise be used to control motor sources 102A and 102B. In one exemplary embodiment, controller 104 can include a vibration intensity controller such as a rheostat, an on-off control and other suitable circuitry. Bandage 108 can comprise an elastic wrap bandage or other suitable support structures to position motor sources 102A and 102B over the protected wound. In one exemplary embodiment, micro-circuitry can be disposed in single device that contains the vibration motor with all controls as described as well as a bulk dressing. In another exemplary embodiment, motor sources 102A and 102B can be positionable on bandage 108, such as by providing connector devices, by including pockets or flaps on bandage 108, or in other suitable manners. Connectors 106A and 106B can be Velcro, buttons, zippers or other suitable connectors that allow bandage 108 and motor sources 102A and 102B to be placed in the vicinity of a wound, so as to provide vibrational stimulation to the wound.

In another exemplary embodiment, motor sources 102A and 102B can have wireless interface devices and local vibrational intensity controllers, so as to allow them to receive wireless control data to adjust the vibrational level intensity. Likewise, additional metering and control functionality can be provided directly within motor sources 102A and 102B where a wireless control interface is provided.

FIG. 2 is a diagram of a controller 200 for a vibrational wound dressing, in accordance with an exemplary embodiment of the present disclosure. Controller 200 includes controller 104 and power monitor 202, motor level control 204, kinetic charger 206, user interface 208 and wireless interface 210, each of which can be implemented in hardware or a suitable combination of hardware and software, and which can be one or more software systems operating on a processor and associated devices.

Power monitor 202 provides power monitoring functionality for a power source for vibrational motors. In one exemplary embodiment, power monitor 202 can monitor the voltage and current of a battery to determine the battery discharge characteristics and to compare those discharge characteristics to a stored discharge curve that can be used to estimate a remaining charge. In another exemplary embodiment, power monitor 202 can continuously measure voltage and current and can add or deduct an associated amount of energy from a data register, where the register is calibrated to the energy storage capacity of a battery. Other suitable power monitoring circuitry can also or alternatively be used.

Motor level control 204 provides a motor control signal to increase or decrease an intensity or level of activity of the motor. In one exemplary embodiment, motor level control 204 can increase or decrease a voltage level, where the vibrational intensity of a motor is linearly related to the provided voltage. In another exemplary embodiment, a frequency of a signal applied to a gate of a switching device can be increased or decreased to adjust a frequency of vibration and associated energy of vibration, or other suitable processes can also or alternatively be used. In one exemplary embodiment, motor level control can implement one or more of the following cycles for at least four vibrational devices:

| | Vibrational | Vibrational | Vibrational | Vibrational |
|---|---|---|---|---|
| | device 1 | device 2 | device 3 | device 4 |
| Cycle 1 | On X sec., | On X sec., | On X sec., | On X sec., |
| | off X sec., | off X sec., | off X sec., | off X sec., |
| | repeat | repeat | repeat | repeat |
| Cycle 2 | On X sec., | On X sec., | On X sec., | On X sec., |
| | off X sec., | off Y sec., | off X sec., | off Z sec., |
| | repeat | repeat | repeat | repeat |
| Cycle 3 | On X sec., | On Y sec., | On Z sec., | On X sec., |
| | off Y sec., | off X sec., | off X sec., | off Z sec., |
| | repeat | repeat | repeat | repeat |
| Cycle 4 | On W sec., | On X sec., | On Y sec., | On Z sec., |
| | off W sec., | off X sec., | off Y sec., | off Z sec., |
| | repeat | repeat | repeat | repeat |
| Cycle 5 | On W sec., | On Y sec., | On W′ sec., | On Y′ sec., |
| | off X sec., | off Z sec., | off X′ | off Z′ |
| | repeat | repeat | sec., | sec., |
| | | | repeat | repeat |
| Cycle 6 | Cycle at | Cycle at | Cycle at | Cycle at |
| | frequency X | frequency Y | frequency X | frequency Y |
| Cycle 7 | Cycle at | Cycle at | Cycle at | Cycle at |
| | frequency W | frequency X | frequency Y | frequency Z |
| Cycle 8 | Random | Cycle at | Random | Cycle at |
| | | frequency X | | frequency X |

In these exemplary embodiments, the variables W, X, Y, Z, W′, X′, Y′ and Z′ can have values in seconds, minutes, hertz or other suitable units. Likewise, other suitable variations in vibration and numbers of devices can also or alternatively be used to maximize the generation of plasmin at the wound. In one exemplary embodiment, the location of each vibrational device can be used to determine the operational cycle, where an effected area of each vibrational device is used to determine an amount of vibrational energy and an operational cycle. In another exemplary embodiment, the vibrational cycle and amount of vibrational energy provided can be controlled as a function of tissue type, where a first type of tissue such as fatty tissue may result in the release of the autologous PA from the local endothelial vasculature at a first response characteristic to applied vibrational energy and where a second type of tissue such as muscle tissue may result in the release of the autologous PA from the local endothelial vasculature at a second response characteristic to applied vibrational energy.

Kinetic charger 206 converts mechanical energy into electrical energy, such as by using a linear electric generator that moves a permanent magnet along a linear track through a coil of wire, and by inverting the resulting AC current and using the inverted AC signal to power a battery charging device. Likewise, other suitable processes can also or alternatively be used.

User interface 208 generates data displays for a user and receives user controls. In one exemplary embodiment, user interface 208 can include a touch screen controller that generates a user-selectable display and which receives user-selected data, such as by detecting a location or locations on the display that a user has touched and by correlating those locations to associated data inputs, or in other suitable manners.

Wireless interface 210 receives and transmits wireless data to a remote unit, such as by using Blue Tooth, an IEEE 802.xx standard or other suitable wireless data standards. In one exemplary embodiment, a low power alert or other suitable data can be transmitted to a central alarm unit, such as an application operating on a cell phone.

FIG. 3 is a diagram of an algorithm 300 in accordance with an exemplary embodiment of the present disclosure. Algorithm 300 can be implemented in hardware or a suitable combination of hardware and software, and which can be one or more software algorithms operating on a processor and associated devices.

Algorithm 300 begins at 302, where it is determined whether one or more motors have been attached to a support. In one exemplary embodiment, each motor can be configured to generate a signal when it is properly attached. In another exemplary embodiment, a vibrational dressing can generate a user interface prompt for a user to confirm that the motors have been attached, or other suitable processes can also or alternatively be used. The algorithm then proceeds to 304.

At 304, a support for the motors is positioned around a wound, such as by securing a fastener of a bandage on which the motors are installed, or in other suitable manners. In one exemplary embodiment, each motor can include one or more sensors that generate an enable signal when the motor is secured by a sufficient force, or other suitable processes can also or alternatively be used. The algorithm then proceeds to 306.

At 306, a level intensity is programmed into a controller. In one exemplary embodiment, the level intensity can be programmed by selecting a level intensity from a digital interface, such as by increasing or decreasing a control setting on a user interface or in other suitable manners. The algorithm then proceeds to 308.

At 308, one or more vibrational motors are activated, such as by applying a predetermined DC voltage and current, by applying a predetermined frequency of AC voltage and current or in other suitable manners. The algorithm then proceeds to 310.

At 310, it is determined whether an alarm signal has been received. In one exemplary embodiment, the alarm indicator can indicate a low power level, a loose bandage or other conditions that interfere with the efficacy of the vibrational dressing. If there is no alarm signal, the algorithm returns to 308, otherwise the algorithm proceeds to 312 where an indicator is generated. In one exemplary embodiment, the indicator can be a flashing light, a beeping sound or other suitable indicators. The algorithm then proceeds to 314, where an indicator is transmitted to a monitor, such as by using a wireless interface.

In one embodiment of the present disclosure, a wound dressing is provided that includes one or more vibrational devices configured to generate a mechanical vibration to stimulate the wound healing process. The measurement of vibration acceleration in meters per second squared (m/s2) and the direction and range of the vibrational exposure can be in a suitable range. A controller is coupled to the one or more vibrational devices and is configured to activate and deactivate the vibrational devices. A programmable controller is coupled to the one or more vibrational devices and is configured to activate and deactivate the vibrational devices. A bandage is coupled to the vibrational devices and is configured to secure the vibrational devices adjacent to the wound. A secondary bandage is coupled to the vibrational devices and is configured to secure the vibrational devices adjacent to the primary dressing overlying a wound. A primary or secondary bandage is coupled to the vibrational devices and is configured to deliver vibrational energy to the wound and periwound area on a continuous basis. A primary or secondary bandage is coupled to the vibrational devices and is configured to deliver vibrational energy to the wound and periwound area on an intermittent or programmed basis.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1. A wound dressing, comprising:
one or more vibrational devices configured to generate a mechanical vibration to stimulate a wound healing process of a wound;
a programmable controller coupled to the one or more vibrational devices and configured to activate and deactivate the vibrational devices; and
a bandage coupled to the vibrational devices and configured to secure the vibrational devices adjacent to the wound, wherein the programmable controller is configured to activate and deactivate the vibrational devices in order to maximize a generation of plasmin at the wound.

2. The wound dressing of claim 1 further comprising a secondary bandage coupled to the vibrational devices and configured to secure the vibrational devices adjacent to a dressing overlying a wound so as to stimulate endovascular endothelium.

3. The wound dressing of claim 1 further comprising a bandage coupled to the vibrational devices, the vibrational devices configured to deliver vibrational energy to blood vessel cells that line blood vessels adjacent to the wound and periwound area on a continuous basis.

4. The wound dressing of claim 1 further comprising a bandage coupled to the vibrational devices, the vibrational devices configured to deliver vibrational energy to the wound and periwound area on an intermittent or programmed basis.

5. The wound dressing of claim 1 wherein the programmable controller is configured to activate and deactivate the vibrational devices in order to maximize a generation of plasmin at the wound by stimulation of endovascular endothelium.

6. The wound dressing of claim 1 wherein the programmable controller is configured to activate and deactivate each of the vibrational devices based on a first cycle to stimulate endovascular endothelium adjacent to the wound.

7. The wound dressing of claim 1 wherein the programmable controller is configured to activate and deactivate each of the vibrational devices based on a different cycle for each vibrational device.

8. The wound dressing of claim 1 wherein the programmable controller is configured to activate and deactivate a first and third vibrational device based on a first cycle, a second vibrational device based on a second cycle, and a fourth vibrational device based on a third vibrational cycle.

9. The wound dressing of claim 1 wherein the programmable controller is configured to activate and deactivate a first and second vibrational device based on a first tissue type and a second tissue type.

10. The wound dressing of claim 9 wherein the first tissue type is fatty tissue.

11. The wound dressing of claim 9 wherein the first tissue type is muscle tissue.

12. The wound dressing of claim 9 wherein the first tissue type is fatty tissue and the second tissue type is muscle tissue.

13. The wound dressing of claim 1 wherein the programmable controller is configured to controllably increase or decrease an affected area associated with each vibrational device in order to stimulate endovascular endothelium adjacent to the wound.

14. The wound dressing of claim 1 wherein the programmable controller is configured to controllably increase or decrease an affected area associated with each vibrational device as a function of vibration cycle.

15. The wound dressing of claim 1 wherein the programmable controller is configured to controllably increase or decrease an affected area associated with each vibrational device as a function of vibration energy.

16. A wound dressing, comprising:
one or more vibrational devices configured to generate a mechanical vibration to stimulate a wound healing process of a wound;
a programmable controller coupled to the one or more vibrational devices and configured to activate and deactivate the vibrational devices; and
a bandage coupled to the vibrational devices and configured to secure the vibrational devices adjacent to the wound while protecting the wound.

17. A wound dressing, comprising:
one or more vibrational devices configured to generate a mechanical vibration to stimulate a wound healing process of a wound;
a programmable controller coupled to the one or more vibrational devices and configured to activate and deactivate the vibrational devices; and
a bandage coupled to the vibrational devices and configured to secure the vibrational devices adjacent to the wound while covering the wound.

18. The wound dressing of claim 17 wherein the programmable controller is configured to activate and deactivate a first vibrational device based on a first tissue type having a first release characteristic of autologous plasminogen activator (PA) from a local endothelial vasculature.

19. The wound dressing of claim 18 wherein the programmable controller is configured to activate and deactivate a second vibrational device based on a second tissue type having a second release characteristic of autologous PA from a local endothelial vasculature.

20. The wound dressing of claim 19 wherein the first vibrational device and the second vibrational device are adjacent to each other on the bandage.
